(19) United States
(12) Patent Application Publication
(10) Pub. No.: US 2006/0229690 A1
(43) Pub. Date: Oct. 12, 2006

(54) Device that emits laser beams at automatically-changing pulse frequencies

(76) Inventors: Steven C. Shanks, Mesa, AZ (US); Charles Shanks, Mesa, AZ (US); George Leger, Mesa, AZ (US)

Correspondence Address: ETHERTON LAW GROUP, LLC, 5555 E. VAN BUREN STREET, SUITE 100, PHOENIX, AZ, 85008, US

(21) Appl. No.: 11/443,980

(22) Filed: May 28, 2006

Related U.S. Application Data
(63) Continuation-in-part of application No. 10/612,504, filed on Jul. 1, 2003.
Application No. 10/612,504 is a continuation-in-part of application No. 09/932,907, filed on Aug. 20, 2001, now Pat. No. 6,746,473.
(60) Provisional application No. 60/273,282, filed on Mar. 2, 2001.

(51) Int. Cl. A61N 5/06

(52) U.S. Cl. 607/89

(57) ABSTRACT

An improved hand-held laser device that automatically changes the pulse frequency while the laser beam is being generated. The device comprises one or more laser energy sources and a frequency controller to change the pulse frequency of the emitted laser light. In certain embodiments, the frequency change is predictable and can be defined by a formula. In other embodiments, the frequency change is unpredictable and is determined using a pseudo-random number generator algorithm.

### CROSS-REFERENCE TO RELATED APPLICATIONS

This application claims the benefit of co-pending U.S. application Ser. No. 10/612504 filed Jul. 1, 2003, which claims the benefit of U.S. application Ser. No. 09/932,907 filed Aug. 20, 2001, now issued as U.S. Pat. No. 6,746,473, which claims the benefit of U.S. Provisional Application No. 60/273,282 filed Mar. 2, 2001.

### FIELD OF INVENTION

This invention relates generally to medical devices that employ lasers. More particularly, this invention relates to a laser light generator device that emits laser energy at changing pulse frequencies.

### BACKGROUND

Low level energy laser therapy (LLLT) is used in the treatment of a broad range of conditions. LLLT improves wound healing, reduces edema, and relieves pain of various etiologies, including successful application post-operatively to liposuction to reduce inflammation and pain. LLLT is also used during liposuction procedures to facilitate removal of fat by causing intracellular fat to be released into the interstice. It is also used in the treatment and repair of injured muscles and tendons.

LLLT utilizes low level laser energy, that is, the treatment has a dose rate that causes no immediate detectable temperature rise of the treated tissue and no macroscopically visible changes in tissue structure. Consequently, the treated and surrounding tissue is not heated and is not damaged. There are a number of variables in laser therapy including the wavelength of the laser beam, the area impinged by the laser beam, laser energy, the frequency at which the laser beam is pulsed, treatment duration and tissue characteristics. The success of each therapy depends on the relationship and combination of these variables. For example, liposuction may be facilitated with one regimen utilizing a given wavelength, pulse frequency, and treatment duration, whereas pain may be treated with a regimen utilizing a different wavelength, pulse frequency and treatment duration, and inflammation yet a third regimen. Specific devices are known in the art for each type of therapy.

It has been found that, in some situations, the body's response to a given pulse frequency diminishes over time. In essence, the body adapts to the specific pulse frequency. To combat this problem, it has been found that the body will respond better if laser emissions with changing pulse frequencies are applied so that the body does not readily adapt. Therefore, it is desirable to apply the laser energy with changing pulse frequencies, with the change occurring in a predictable pattern as well as a random fashion.

Programmable laser devices are known in which a first frequency can be programmed for a first period of time, and a second frequency can be programmed for a second period of time, etc., such that the patient can be treated with multiple frequencies in a single application of the laser light. In order for these lasers to properly function, each pulse frequency must be pre-programmed into the device, often manually by the practitioner. It is tedious to program more than a handful of pre-set frequency changes into a single laser program. It would be desirable to have a laser device that emits different pulse frequencies without pre-programming each frequency. Moreover, the lasers in the prior art are not capable of emitting random pulse frequencies, because it is nearly impossible for a human to program the frequencies in a random manner. It would be desirable to have a laser device that emits random pulse frequencies without pre-programming each frequency.

Therefore, an object of this invention is to provide a laser device that automatically changes the pulse frequency of at least one laser beam while the beam is being generated. It is yet a further object of the present invention to provide a laser device that automatically changes the pulse frequency in a predictable fashion while the beam is being generated. It is another object of this invention to provide a laser device that automatically changes the pulse frequency in an unpredictable fashion while the beam is being generated.

### SUMMARY OF THE INVENTION

This invention is an improved hand-held laser device that automatically changes the pulse frequency while the laser beam is being generated. The device comprises one or more laser energy sources and a frequency controller to change the pulse frequency of the emitted laser light. In certain embodiments, the frequency change is predictable and can be defined by a formula. In other embodiments, the frequency change is unpredictable and is determined using a pseudo-random number generator algorithm.

### BRIEF DESCRIPTION OF THE DRAWINGS

FIG. 1 is a schematic illustration of the present invention.

FIG. 2 is a schematic illustration of the preferred embodiment of the present invention.

FIG. 3 is a schematic illustration of a second embodiment of the present invention with more than one laser energy source.

FIG. 4 is a schematic illustration of another embodiment of the present invention with more than one laser energy source and a single control circuit controlling the pulse frequency.

FIG. 5 is a schematic illustration of a second preferred embodiment of the present invention with multiple hand-held probes each containing a laser energy source, a control circuit, and an optical arrangement.

FIG. 6 is a schematic illustration of an alternate embodiment of the present invention, with a single laser source that is split into multiple beams.

FIG. 7 is a schematic illustration of application of low-level laser radiation using the preferred embodiment of the present invention.

FIG. 8a is a block diagram illustrating some of the circuitry comprising a control circuit.

FIG. 8b is a block diagram illustrating one method of producing random pulse frequencies in the present invention.

FIGS. 9a-f are graphs illustrating the frequency-vs-time curves for several predictable and non-predictable laser outputs.

FIG. 10 is schematic of the frequency controller using shift registers.

### DETAILED DESCRIPTION OF THE INVENTION

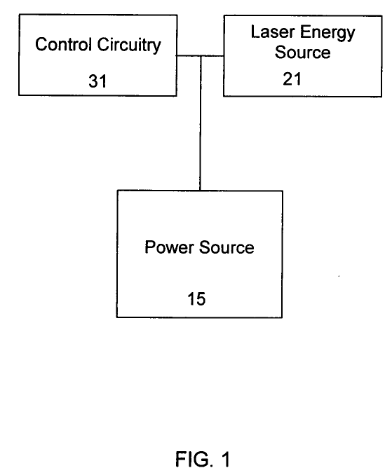

The present invention comprises at least one laser energy source 21, control circuitry 31 to control parameters of the laser beams emitted from the laser energy source 21, and a power source 15. See FIG. 1. Any laser energy source 21 of any wavelength can be used, including those of visible, infrared, and ultraviolet wavelengths. Preferably, physically small laser energy sources such as semiconductor laser diodes are used. These are preferably battery powered and disposed in the probe itself. The batteries maybe rechargeable. Alternatively, the batteries may be in a base that recharges the device when the device is in connection with the base. Larger laser energy sources may also be used. These are typically housed in a base and the laser light is carried on fiber optic lines to a probe from which the laser beam emits. The base is typically a stationary unit that sits on a table or the ground, functioning as a central base from which many probes may be employed. Alternatively, the base may be a hand-held unit that houses various controls. A Helium-Neon laser having a 632 nm wavelength is an example of a larger laser source.

Preferably the laser energy source 21 is a low-level laser energy source, such as those known in the art for low-level laser therapy, which has a dose rate that causes no immediate detectable temperature rise of the treated tissue and no macroscopically visible changes in tissue structure. Visible light in about the 400-800 nm range is preferred. The laser energy source 21 in the preferred embodiment is a semiconductor laser diode that produces light in the red range of the visible spectrum, more preferably having a wavelength of about 635 nm. Other suitable wavelengths are used for other particular applications. While many LLLT regimen include visible laser light, it may be advantageous to utilize ultraviolet or infrared laser energy, again depending on the type of treatment desired. Solid state and tunable semiconductor laser diodes may also be employed to achieve the desired wavelength. Different therapy regimens require diodes of different wattages. The preferred laser diodes use less than one watt of power each.

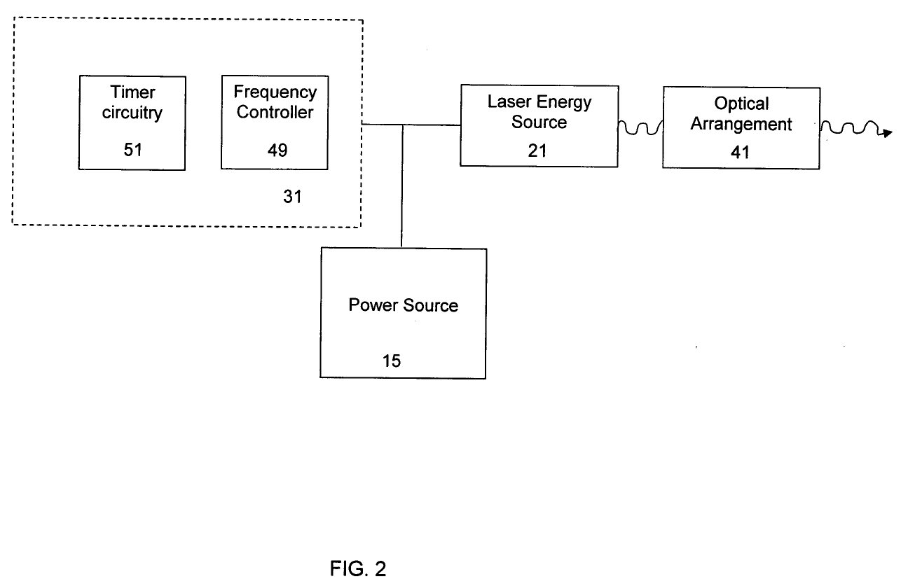

In the preferred embodiment, as shown in FIG. 2, the laser energy source 21 emits a laser beam that is transmitted through an optical arrangement 41 to modify the beam spot. The beam spot is the cross-sectional shape and size of the emitted beam as it exits the optical arrangement 41. For example, a laser beam of circular cross-section creates a circular beam spot as the laser light impinges the patient's skin. If the laser light emitted is in the visible range, a circular spot can be seen on the patient's skin of substantially the same diameter as the laser beam emitted from the optics arrangement. Various beam spot shapes can be created, including a line, a rectangle, a circle, an ellipse, a plus-sign, or combination thereof. If multiple laser beams are emitted, they may have different spot shapes, or have the same spot shapes. Optical arrangement 41, as used herein, includes any arrangement of one or more optical, mechanical or electrical or other parts. For example, to generate a linear beam spot, the laser beam may be passed through a collimating lens and a line generating prism. Or, it can be passed through a collimating lens and a rod lens. Other optical arrangements for generating a linear beam spot include a diffraction grating, mechanical slit, or other optical arrangement 41.

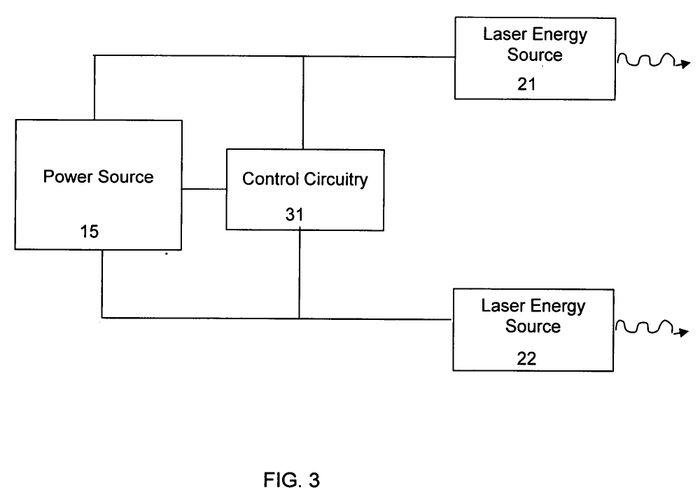

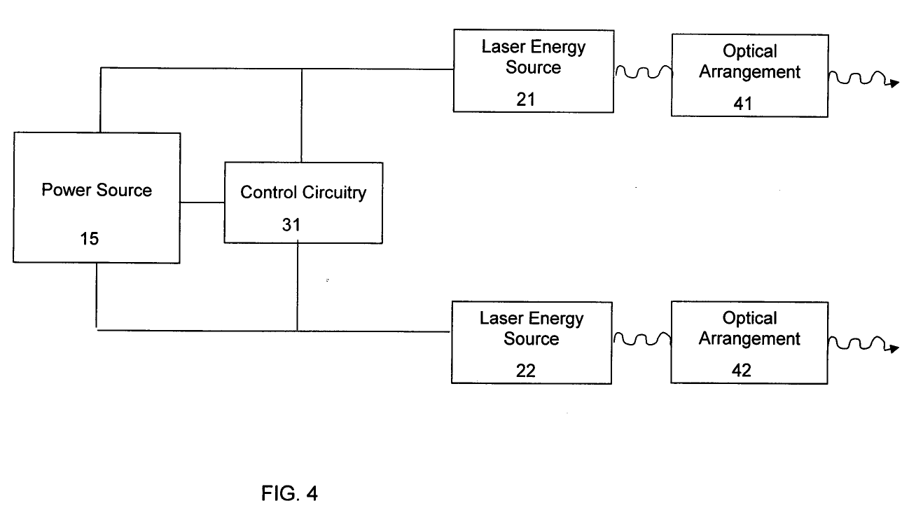

The device may comprise more than one the laser energy source 21. FIG. 3 illustrates an embodiment with two the laser energy sources 21, 22 sharing the control circuitry 31. Alternately, each the laser energy source 21 can have its own control circuitry 31. More than two the laser energy sources 21, 22 can be used; commonly four are used. FIG. 4 shows a multi-source device with a shared control circuitry 31 and optical arrangements associated with each the laser energy source 21.

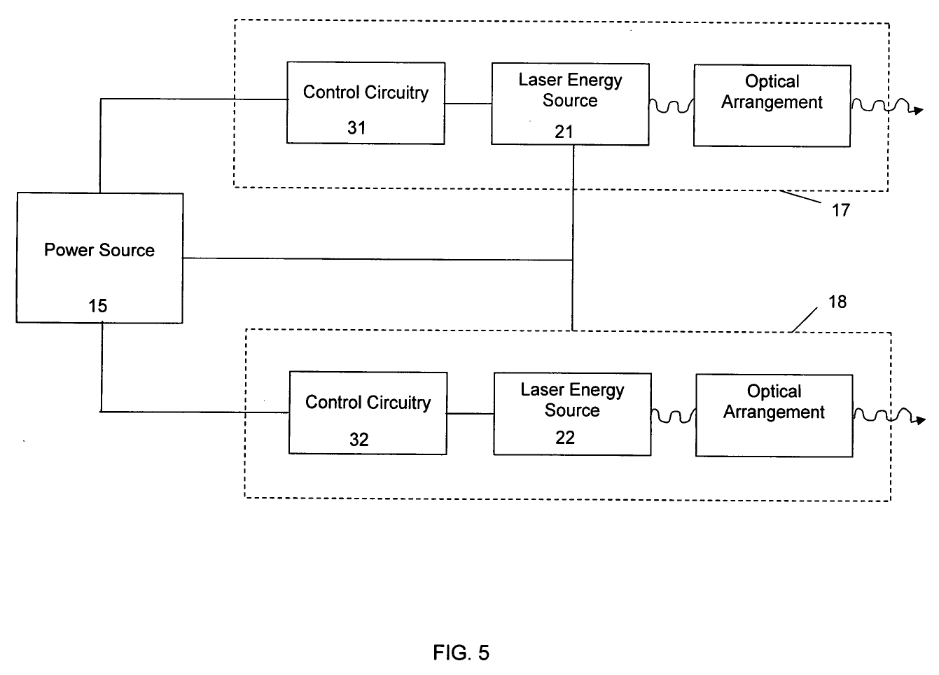

FIG. 5 illustrates a second preferred embodiment of the present invention which has multiple probes. Dotted line 17 indicates the components disposed in a first probe and dotted line 18 indicates the components disposed in a second probe. Each probe has its own laser energy source 21, 22; an optical arrangement 41, 42; and the control circuitry 31, 32, respectively. The probes share the same power source 15. Each probe could be equipped with its own power source 15 if desired. In this embodiment, the pulse frequency of laser light emitted from the first probe is distinct from the pulse frequency of laser light emitted from the second probe. Other embodiments may emit laser light at the same pulse frequency from each probe. Light can be emitted simultaneously from both probes or from only one probe at a time.

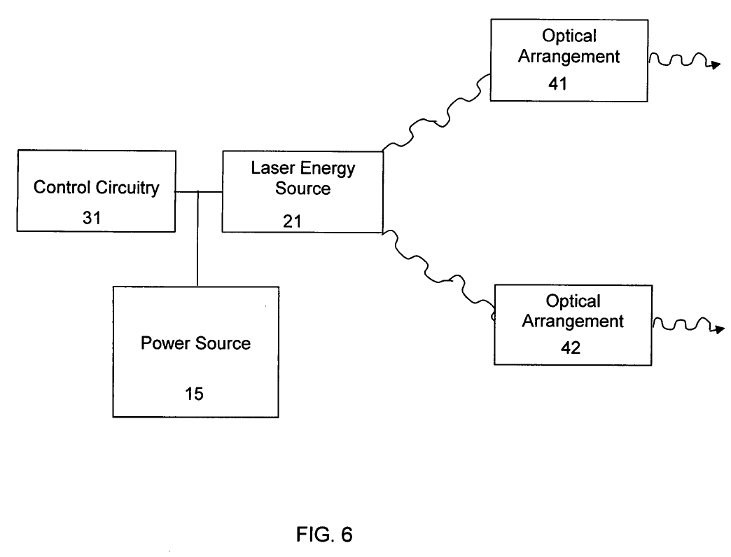

In another embodiment shown in FIG. 6, the laser energy source 21, power source 15, and the control circuitry 31 are disposed in a single hand-held probe, and the laser beam is split to go through more than one optical arrangements 41, 42.

Power source 15 preferably provides direct current, such as that provided by a battery, but may instead provide alternating current such as that provided by conventional building current which is then converted to direct current. Laser energy source 21 can be energized independently or simultaneously, which throughout this specification refers to acts occurring at generally at the same time.

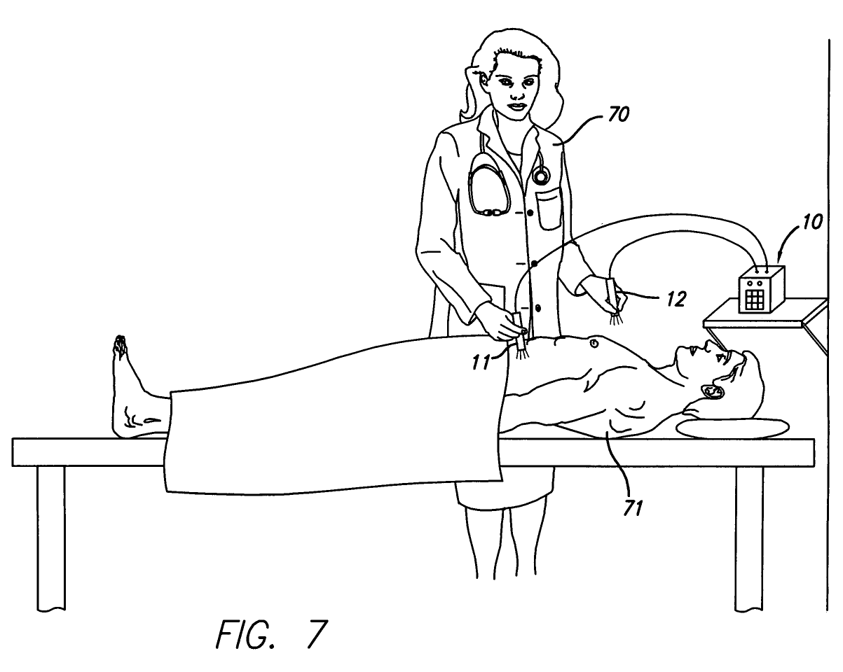

The control circuitry connected to the laser energy source 21 controls the various parameters of the emissions, such as to control whether each laser is powered on, how long each laser generates a laser beam, or the pulse frequency of the emitted beam. Typically the control circuitry is digital, in discrete or integrated circuits, as is known in the art, but analog circuits can also be employed. In the preferred embodiment, separate control circuits 31, 32 are used for each probe. See FIG. 5. These control circuits are operatively connected to the laser energy sources 21, 22, respectively. FIG. 7 illustrates the device in use. A practitioner 70 treats a first area of the patient 71 with a first probe 11 and treats a different area of the patient 71 with a second probe 12.

For ease of reference, pulse frequencies can be referred to in shorthand notation in cycles/second, or Hz. Pulse frequencies from 0 to 100,000 Hz may be employed to achieve the desired effect on the patient's tissue. At 100,000 Hz, the pulse cycle is 0.00001 second. At 0 Hz, a continuous beam of laser light is generated.

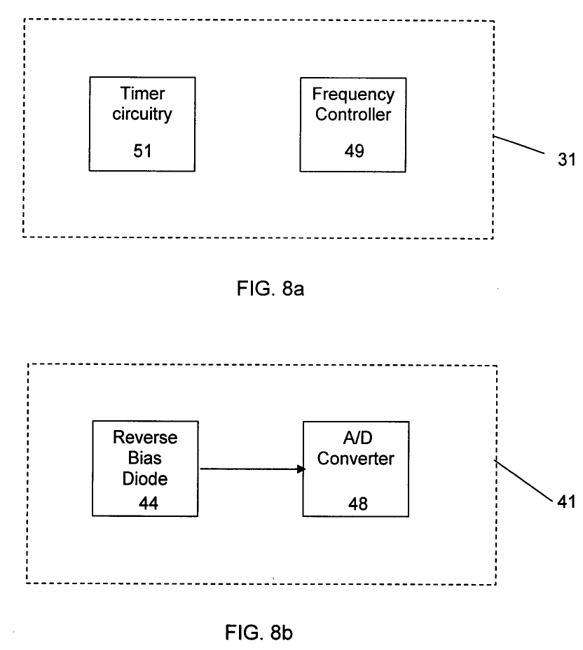

The frequency controller 49 operatively communicates with one or more laser energy sources 21. FIG. 8a illustrates the frequency controller 49 and the timer circuitry 51 logically included in the control circuitry 31, which in turn is operatively connected to the laser energy source 21. In the preferred embodiment, the frequency controller 49 is a microcontroller which includes at least a CPU and memory. The electronic controller stores one or more patterns in memory, preferably in the form of a formula, although discrete values may be stored. The frequency controller 49 uses the formula to generate one or more laser beams with a pulse frequency that changes in accordance with a pattern that is predicted by the formula.

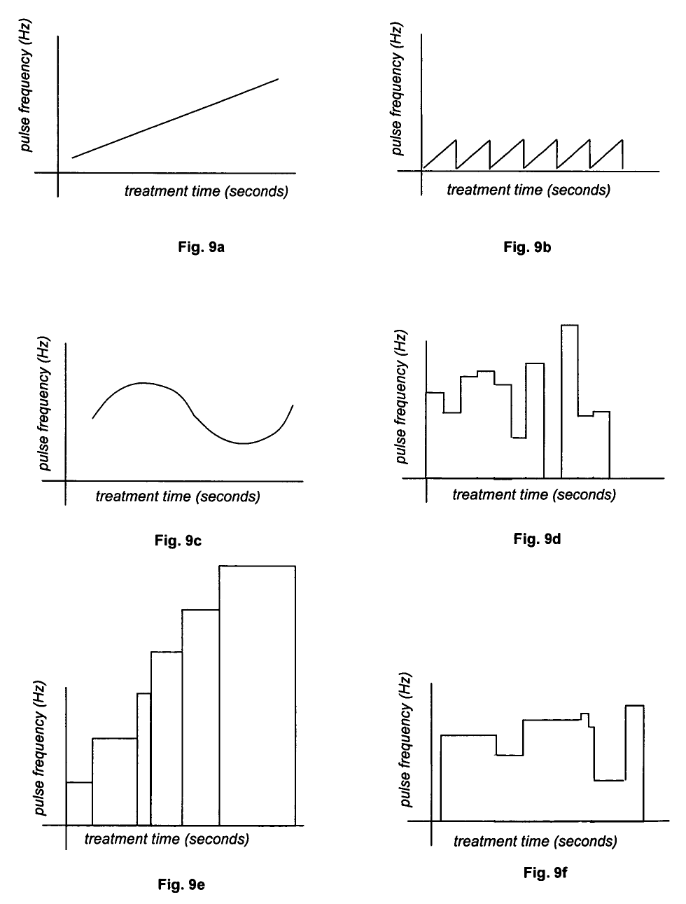

These predictable changes include patterns that are linear, saw tooth, stepped, sinusoidal, exponential, Gaussian, bell-shaped, or shaped otherwise. See FIGS. 9a-f. For example, during a linear increase, as shown in FIG. 9a, the laser energy source 21 may initially emit light with a pulse frequency of 10 Hz and increase the pulse frequency by 1 Hz every second until it the treatment time expires. In an example of a saw tooth pattern, as shown in FIG. 9b, the laser energy source 21 may initially emit light with a pulse frequency of 1 Hz and increase the pulse frequency 1 Hz every second until it reaches 50 Hz. Once it reaches the 50 Hz threshold, the laser energy source 21 may again begin to emit light with a pulse frequency of 1 Hz and begin its linear climb to 50 Hz again. Of course, the rate of change, upper and lower limits, and time at each frequency may change. FIG. 9c illustrates sinusoidal pulse frequency pattern.

In other embodiments, the change in pulse frequency is substantially random. Random, as used herein, is defined as substantially unpredictable and having outputs each of which have the equal chance of being selected from all potential outputs. The outputs of pseudorandom number generators are not truly random—they only approximate some of the properties of random numbers. However, for practical applciations the pseudo-randomness suffices. Common classes of pseudo-random number generator algorithm are the Lagged Fibonacci, Blum Blum Shub, Fortuna, and the Mersenne Twister. Any algorithm or method to generate a substantially random pulse frequency can be used and fall within the scope of the present invention.

Random pulse frequencies can be produced by numerous software and hardware methods, and combinations thereof. In another preferred embodiment, the frequency controller 49 is again a microcontroller which includes at least a CPU and memory. A pseudo-random number generator algorithm is stored in memory which generates a sequence of numbers that are substantially random. The sequence is initiated with a randomly generated “seed number” or initial number. The seed value may be generated continuously and is loaded into the controller whenever the user of the laser initiates the random pulse frequency.

To generate a truly random seed, it is known to convert the date and time to an integer value (for example, convert the current date and time to the number of seconds that have elapsed since Jan. 1, 1970). Because time changes continuously, the resultant value is a different number every time. For example, the random pulse frequency may be initiated by the user pressing a “start” button. Depressing the start button initiates the generation of the seed value. Because the exact time the start button (or other similar control device) is pressed can never be the same, the number generated is random. The seed values are then used in the pseudo-random number generator algorithm to generate random numbers to set the frequency of the frequency controller 49. The device is then capable of producing a laser beam that pulses at a substantially random frequency.

Other algorithms for generating random seeds are known, such as mathematical algorithms. For example, a seed value set by time and date, as above, is to multiplied by 1,103,515,245 and then added to 12,345 to obtain the next seed number. The random seed is then replaced by this new value to generate the next seed, and so on.

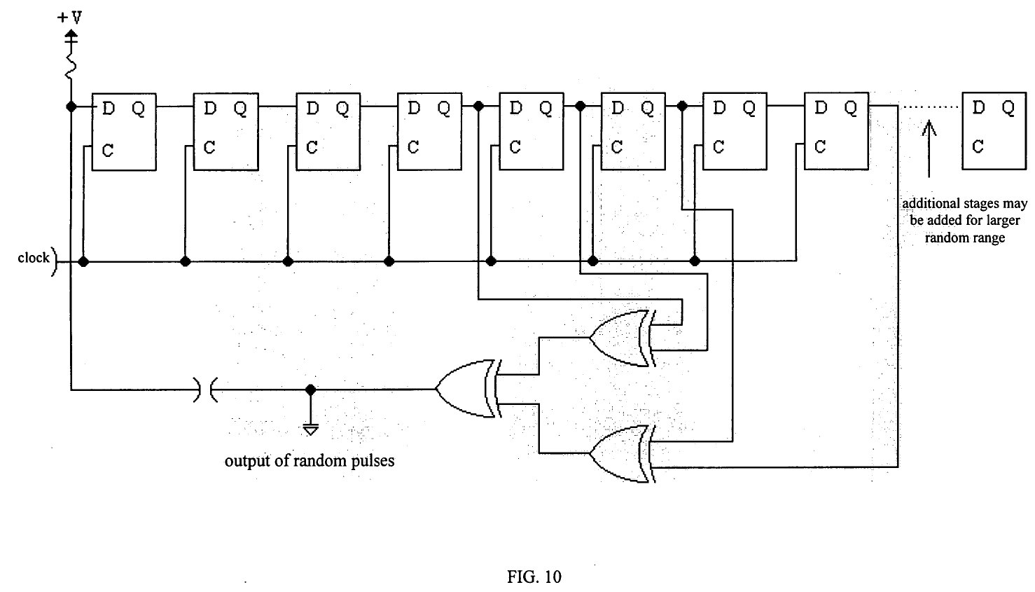

Another embodiment can be implemented in hardware by using a series of shift registers. A random seed is generated, and the shift registers are connected in a feedback arrangement such that the outputs produce nearly ten million random frequencies that can be used to modulate the laser energy source 21. The preferred system comprises a multi-stage shift register circuit configuration with the appropriate feedback to generate a maximal length sequence. See FIG. 10.

An electrical thermal noise source is considered to produce one of the most random outputs in nature. By sampling a noise source voltage at any given time one can create a random occurrence to be used as a seed. In another embodiment, a white noise generator circuit is amplified and used to modulate the laser diodes or other laser energy source 21. Noise diodes are avalible commercially, for example for Micronetics of Hudson, N.H. In yet another embodiment, a reversed bias diode 44 acts as a noise generator that produces white noise. See FIG. 8b. The noise is amplified and fed into an analog-to-digital converter 48. In one embodiment, A/D converter 48 is a 12 bit A/D converter, but other A/D converters can be used and fall within the scope of the present invention. The value output from A/D converter 48 is sampled randomly and the resultant value is used to in the frequency controller 49 in conjunciton with the laser energy source 21 to produce random pulse frequencies.

Whether generated by hardware or software, in the preferred embodiment the resultant random number is generated and used by the controller as the random seed. The pulse frequencies of the diodes of the preferred embodiment are changed at one cycle per second and, as the new random number is loaded into the controller every cycle, a new frequency is generated every second. While specific examples have been discussed above to enable the laser device to produce random pulse frequencies, it should be noted that other known methods and devices to generate random pulse frequencies can be used and fall within the scope of the present invention.

The device can be operated in pattern mode, random mode, or combinations thereof. In one embodiment, the laser device has two modes: one mode to emit random pulse frequencies and another to emit patterned pulse frequencies. In another embodiment, the laser device can operate whereby the pulse frequency changes in accord with a pattern for a given period of time before the pulse frequency is emitted randomly for a set period of time. For example, a laser device might emit light with a pulse frequency that changes in accord with a pattern for ten seconds and then changes randomly for ten seconds. This change between patterned and random pulse frequency can repeat itself.

In addition to patterned or random changes in frequency, patterned or random changes in the timing can be similarly controlled. The timer 51 operatively communicates with one or more laser energy sources 21. FIG. 8a illustrates the frequency controller 49 and the timer circuitry 51 logically included in the control circuitry 31, which in turn is operatively connected to the laser energy source 21. Similar to the frequency controller, the timer 51 can cause the period of time to change in a pattern or randomly, as explained above. In one embodiment, the pulse frequency changes randomly, but the output laser beam is held at the randomly-selected frequency for a set period of time. This would result in, for example, a randomized step function. See FIG. 9d. In another embodiment, the set period of time could be randomized while the pulse frequency changes in a pattern. See FIG. 9e. In yet another embodiment, both the time the laser is on as well as the pulse frequency it operates at are randomized. See FIG. 9f.

While there has been illustrated and described what is at present considered to be a preferred embodiment of the present invention, it will be understood by those skilled in the art that various changes and modifications may be made, and equivalents may be substituted for elements thereof without departing from the true scope of the invention. Therefore, it is intended that this invention not be limited to the particular embodiment disclosed as the best mode contemplated for carrying out the invention, but that the invention will include all embodiments falling within the scope of the appended claims.

We claim:

1. A laser device comprising:
a) one or more laser energy sources for generating one or more laser beams;
b) one or more frequency controllers in operative communication with at least one laser energy source;
c) wherein at least one frequency controller automatically changes the pulse frequency of at least one laser beam while the laser beam is being generated.

2. A device according to claim 1 wherein the pulse frequency of at least one laser beam changes substantially randomly.

3. A device according to claim 2 wherein at least one frequency controller further comprises a pseudorandom number generator algorithm.

4. A device according to claim 2 wherein at least one frequency controller further comprises a random number generator.

5. A device according to claim 4 wherein at least one frequency controller further comprises a noise generator circuit.

6. A device according to claim 2 wherein at least one frequency controller further comprises one or more shift registers.

7. A device according to claim 1 in which the pulse frequency changes every second.

8. A device according to claim 1 wherein the pulse frequency changes linearly.

9. A device according to claim 1 wherein the pulse frequency changes sinusoidally.

10. A device according to claim 1 wherein the pulse frequency changes in a step function.

11. A device according to claim 1 wherein the pulse frequency changes exponentially.

12. A device according to claim 1 wherein the pulse frequency changes follow a substantially Gaussian curve.

13. A device according to claim 1 wherein at least one laser energy source is a semiconductor diode.

14. A device according to claim 1 wherein at least one laser energy source is housed in a base.

15. A device according to claim 1 wherein at least one laser energy source is housed in a hand-held probe.

16. A device according to claim 1 wherein one or more laser beams has a wavelength in the visible range.

17. A device according to claim 16 wherein one or more laser beams is red.

18. A device according to claim 1 wherein one or more laser beams has a wavelength in the infrared range.

19. A device according to claim 1 wherein one or more laser beams has a wavelength in the ultraviolet range.

20. A device according to claim 1 further comprising one or more optical arrangements for receiving one or more laser beams and for transforming each of the laser beams into a desired spot shape.

21. A device according to claim 20 wherein at least one spot shape is substantially linear.

22. A device according to claim 20 wherein at least one spot shape is substantially circular.

23. A device according to claim 20 wherein at least one spot shape is substantially elliptical.

24. A laser device according to claim 1 wherein:
a) the pulse frequency changes in a pattern for a period of time; and
b) the pulse frequency changes randomly for a period of time.

25. A laser device according to claim 1 wherein the pulse frequency changes in a pattern for a random period of time.

26. A laser device according to claim 1 wherein the pulse frequency changes randomly for a random period of time.

27. A laser device comprising:
a) one or more the laser energy sources housed within a hand-held wand for generating one or more laser beams;
b) an optical arrangement for receiving at least one laser beam and for transforming at least one laser beam into a desired spot shape; and
c) one or more the frequency controllers in operative communication with one or more the laser energy sources to generate one or more laser beams with a pulse frequency that changes randomly.

28. A laser device comprising:
a) one or more the laser energy sources housed within a hand-held wand for generating one or more laser beams;
b) an optical arrangement for receiving at least one laser beam and for transforming at least one laser beam into a desired spot shape; and
c) one or more the frequency controllers in operative communication with one or more the laser energy sources to generate one or more laser beams with a pulse frequency that changes in a predictable pattern.
